(19) United States
(12) Patent Application Publication
(10) Pub. No.: US 2005/0214369 A1
(43) Pub. Date: Sep. 29, 2005

(54) Lecithin-containing granular compositions and methods of their preparation

(76) Inventors: Myong K. Ko, Shakopee, MN (US); Harapanahalli S. Muralidhara, Plymouth, MN (US); Heidi Schmitt, Aumuehle (DE); Michael Schneider, Freinsheim (DE); John Van De Sype, Semmerzake (BE)

Correspondence Address: PERKINS COIE LLP, P.O. BOX 2168, MENLO PARK, CA, 94026, US

(21) Appl. No.: 10/992,427

(22) Filed: Nov. 17, 2004

Related U.S. Application Data
(60) Provisional application No. 60/523,192, filed on Nov. 17, 2003.

(51) Int. Cl. A61K 9/24; D04H 1/00

(52) U.S. Cl. 424/471; 264/109

(57) ABSTRACT

Methods of preparing granular compositions containing lecithin, preferably as a major or sole ingredient, are described. Granular lecithin-containing compositions are also described and can be prepared by the disclosed methods.

This application claims priority to U.S. Provisional Application No. 60/523,192, filed on Nov. 17, 2003, which is incorporated herein in its entirety by reference.

### FIELD OF THE INVENTION

The present invention relates to granular lecithin compositions and methods of preparing such compositions. In particular, the invention relates to methods of preparing granular compositions having large proportions of lecithin, e.g. 30% by weight or more, by roller compaction or fluid bed granulation.

### REFERENCES

Barker T G et al., “Method of Preparing a Granular Free-Flowing Lecithin Product”, U.S. Pat. No. 3,920,857 (November 1975).
Bertelli A, “Pharmaceutical Compositions Having Cerebral Antianoxic and Metabolic Activities”, U.S. Pat. No. 4,684,520 (August 1987).
Gaynor M L and Hickey G P, “Green Nutritional Powder Composition”, U.S. Pat. No. 5,904,924 (May 1999).
Gaynor M L, “Nutritional Powder Composition”, U.S. Pat. No. 5,744,187 (April 1998).
Lantz R A and Rothfuss D, “Method of Tableting of De-Oiled Phosphatides (Lecithin)”, U.S. Pat. No. 4,762,658 (August 1988).
Losch R. et al., “Phospholipid Composition”, U.S. Pat. No. 5,310,734 (May 1994).
Losch R. et al., “Phospholipid Composition”, U.S. Pat. No. 5,438,044 (August 1995).
Orthoefer F T, “Compressed Lecithin Preparations”, U.S. Pat. No. 6,312,703 (November 2001).
Peters S E and Woods D H, “Stable Aqueous Dispersion of Nutrients”U.S. Pat. No. 6,455,072 (September 2002).
Pozzi F et al., “Pharmaceutical Composition Containing Ubidecarenone”, U.S. Pat. No. 4,869,900 (September 1989); PCT Pubn. No. WO 864503 (August 1986).
Stuckler F, “Natural Substances Based Agent”, U.S. Pat. No. 6,605,296 (August 2003).
Venkatesh G M et al., “Process for Manufacturing Bite-Dispersion Tablets”, PCT Pubn. No. WO 99/32092 (July 1999).

### BACKGROUND OF THE INVENTION

Lecithin is widely used as an emulsifier and solubilizer in the food, feed, pharmaceutical and cosmetic industries and has numerous other industrial uses, e.g. as an anti-foaming, dispersing or wetting agent in the manufacture or processing of foods, chemicals, inks, paints, pigments, polymers, textiles, and various other products. It is also known to possess beneficial therapeutic properties when used in a dietary supplement.

Commercial lecithin is generally “deoiled” lecithin, a solid obtained by extracting fluid lecithin with acetone to remove free fatty acids, diglycerides, triglycerides and other neutral lipids. This deoiled lecithin powder is generally a hygroscopic material which compacts readily into a dense, often sticky, mass. The material tends to stick to the surfaces of containers, and when taken orally, to stick to surfaces within the mouth. Accordingly, methods have long been sought for producing solid lecithin in a more easily handleable form.

In one approach, solid particulate formulations have been provided by incorporating dry fillers, such as talc, fused silica, starches, milk solids, and the like into lecithin powder. However, large quantities of such fillers are often undesirable and may interfere with the useful properties of the lecithin. In a pharmaceutical or nutraceutical formulation to be taken orally, they increase the amount of the formulation which must be used to provide a given dosage of the active ingredients.

In current practice, “granular” lecithin is formed by an acetone wet granulation process. This process, however, generally produces substantially less than 50% of actual granular material. The remainder is obtained as a fine powdery material, which has the poor handling and mouth feel characteristics described above. This process is also unsuitable for incorporation of compounds which are soluble in acetone, which are unstable in the acetone extraction environment, or which are otherwise unsuitable for use in this process, into the final product.

Accordingly, methods are desired for efficiently preparing lecithin or lecithin-containing materials in free-flowing, granular form. Ideally, such methods would also be suitable for incorporating a variety of additional components into the granules.

### DETAILED DESCRIPTION OF THE INVENTION

I. Overview

As described more fully below, various embodiments of the invention include granular compositions comprising lecithin and optionally one or more further ingredients, and methods of producing the granular compositions.

Accordingly, the invention includes, in one aspect, a method of producing a granular lecithin-containing composition, the method comprising:
processing in a roller compaction granulator: (i) a solid comprising at least 30% by weight lecithin, typically a deoiled lecithin powder, and an optional further component, with (ii) a binder material, in an amount of about 0.1% to 5% by weight relative to the solid (i), and (iii) an antisticking agent, in an amount of about 0.1% to 5% by weight relative to the solid (i).

In selected embodiments, each of the binder material and antisticking agent is, independently, present in an amount of about 0.2% to 1.5% by weight, relative to the solid (i).

The binder material is preferably selected from the group consisting of sugars, starches, lactates, soy proteins, and hydrophilic polymers, such as cellulose ethers, gums, and polylactates. In selected embodiments, the binder material is a cellulose ether, such as methyl cellulose or hydroxypropyl methylcellulose. The cellulose ether is preferably present in an amount of about 1% by weight relative to the lecithin-containing solid (i).

The antisticking agent is preferably selected from the group consisting of silicon dioxide, calcium silicate, tricalcium phosphate, calcium stearate, zinc stearate, and magnesium stearate. In one embodiment, the antisticking agent is magnesium stearate, which is preferably present in an amount of about 0.5% to 1% by weight relative to the solid (i).

Typically, the lecithin-containing solid (i) contains 30-100% by weight lecithin, which includes pure (100%) lecithin. A further component, if present, may be an active ingredient such as a pharmaceutical or nutraceutical. In further embodiments, the lecithin-containing solid (i) contains at least 50% by weight lecithin, or at least 75% by weight lecithin. The lecithin in the solid to be granulated is typically in powder form.

In a related aspect, the invention provides a granular lecithin-containing composition produced by the above-disclosed granulation method. This composition preferably comprises about 30-99% by weight lecithin, the above-referenced binder, and the above-referenced antisticking agent. The further component, if present, may be an active ingredient such as a pharmaceutical or nutraceutical. In further embodiments, the granular lecithin-containing composition comprises about 50-99% by weight lecithin or about 75-99% by weight lecithin. In one embodiment, the granular composition consists of lecithin, a binder as disclosed above, and an antisticking agent as disclosed above, wherein each of the two latter components is present, independently, in an amount of about 0.1% to 5% by weight, preferably about 0.2% to 1.5% by weight, relative to said lecithin.

In another aspect, the invention provides a further method of producing a granular lecithin-containing composition, the method comprising:
processing in a fluidized bed granulator, at an inlet temperature of at least 40° C., (i) a solid comprising at least about 30% lecithin, typically a deoiled lecithin powder, and an optional further component, with (ii) a solution of a binder material, where the solution has a viscosity effective to provide efficient spraying and atomization of the solution. Preferably, the viscosity of the solution is about 100 to 300 centipoise.

The solution (ii) preferably comprises a solvent selected from the group consisting of water, acetone, and C1-C4 alcohols. In one embodiment, the solvent is water.

In selected embodiments of the method, at least one of the solid (i) and the solution (ii) contains one or more additional components, such as one or more nutraceutical components. When the solution contains one or more additional components, it is preferably present in the solution at a level of about 0.001%-1% by weight relative to the solid (i).

The binder material is preferably selected from the group consisting of sugars, starches, lactates, soy proteins, and hydrophilic polymers, such as cellulose ethers, gums, and polylactates. In selected embodiments, the binder material is a cellulose ether, such as methyl cellulose or hydroxypropyl methylcellulose.

Preferably, the binder solution includes an amount of binder (solids) which is up to 5% by weight, e.g. 0.1-5% by weight, more preferably 0.2-1.5% by weight, relative to the weight of the lecithin-containing material. In selected embodiments, the binder is present at 0.5-1.5% percent by weight (solids) of the lecithin-containing material.

In selected embodiments of the method, the inlet temperature is at least 50° C.; in further embodiments, the inlet temperature is about 40-100° C., or about 50-90° C. The outlet temperature in the granulation process is preferably in the range of about 35-60° C.

The solid (i) to be granulated preferably contains at least 50% by weight lecithin; in further embodiments, the solid contains at least 75% by weight lecithin. In one embodiment, the solid is 100% lecithin. Typically, the lecithin in the solid to be granulated is in powder form.

Either of the above-disclosed methods may further comprise a step of: processing in a fluidized bed granulator, at an inlet temperature of at least 40° C., the granular lecithin-containing composition with a solution of a further component, where the solution has a viscosity effective to provide efficient spraying and atomization of the solution. Preferably, the viscosity is about 100 to 300 centipoise.

In a further aspect, the invention provides a granular lecithin-containing composition produced by the above-disclosed fluidized bed granulation method. Preferably, the composition comprises 30-99.9% by weight lecithin and the above-referenced binder. A further component, if present, may be an active ingredient such as a pharmaceutical or nutraceutical. In further embodiments, the composition comprises about 50-99.5% by weight lecithin, or about 75-99.5% by weight lecithin. In one embodiment, the granular composition consists of lecithin and a binder as disclosed above, wherein the binder is present in an amount of about 0.1% to 5% by weight, preferably about 0.2% to 1.5% by weight, relative to said lecithin.

In a related aspect, the invention provides a granular lecithin-containing composition, comprising:
(i) at least 90% by weight of a preparation comprising lecithin and an optional further component or components, wherein the preparation contains at least 30% lecithin by weight;
(ii) a binder material, in an amount of about 0.1% to 5% by weight, preferably about 0.5% to 2.5% by weight, relative to (i), and, optionally, (iii) an antisticking agent, in an amount of about 0.1% to 5% by weight relative to (i).

In selected embodiments, the granular lecithin-containing composition comprises at least 85%, at least 95%, at least 98%, or at least 99% by weight of the preparation (i). In further embodiments, the preparation (i) is at least 50% by weight lecithin, at least 75% by weight lecithin, or at least 90% by weight lecithin. A further component, if present, may be an active ingredient such as a pharmaceutical or nutraceutical. In one embodiment, the preparation (i) is 100% lecithin.

In a further embodiment, the granular lecithin-containing composition does not include an antisticking agent. When the preparation (i) is 100% lecithin, the composition thus consists of lecithin and the binding agent (ii), preferably in an amount of 0.5% to 2.5%, more preferably 0.5% to 1.5%, by weight relative to the lecithin.

In another further embodiment, the granular lecithin-containing composition does include an antisticking agent. When the preparation (i) is 100% lecithin, the composition thus consists of lecithin, binding agent (ii), and antisticking agent (iii), each of (ii) and (iii) preferably being present, independently, in an amount of 0.5% to 2.5%, more preferably 0.5% to 1.5%, by weight relative to the lecithin.

The binder material (ii) is preferably selected from the group consisting of sugars, starches, lactates, soy proteins, and hydrophilic polymers, such as cellulose ethers, gums, and polylactates. Preferably, the binder is a cellulose ether, such as methyl cellulose or hydroxypropyl methylcellulose.

When the composition includes the antisticking agent (iii), the antisticking agent is preferably selected from the group consisting of silicon dioxide, calcium silicate, tricalcium phosphate, calcium stearate, zinc stearate, and magnesium stearate. More preferably, the antisticking agent is magnesium stearate.

When the preparation (i) contains a further component, the component is, in selected embodiments, one or more nutraceutical substances. The nutraceutical component is typically present at a level of about 0.05% to 40% by weight of the lecithin-containing preparation (i). The nutraceutical component may be selected, for example, from the group consisting of vitamins, phytosterols, phytostanols, creatine, L-carnitine, coenzyme Q 10, lycopene, and lutein. For example, the component(s) may comprise: phytosterols and/or phytostanols, which are preferably present at a level of at most 17% of the preparation (i); creatine, which is preferably present at a level of at most 5% of the preparation (i); L-carnitine, which is preferably present at a level of at most 35% of the preparation (i); coenzyme Q10, which is preferably present at a level of at most 0.5% of the preparation (i); lycopene, which is preferably present at a level of at most 0.1% of the preparation (i); or lutein, which is preferably present at a level of at most 0.1% of the preparation (i). In each case, the remainder of the preparation (i) is preferably lecithin.

These and other objects and features of the invention will become more fully apparent in view of the following detailed description of the invention.

II. Definitions

As used herein with respect to the current invention, e.g. in the appended claims, the terms “granular”, “granulated” or “granules”, used in combination with terms such as lecithin, lecithin-containing product, lecithin composition, lecithin-containing composition, etc., refer to lecithin or lecithin-containing material that has been granulated by a granulation process as disclosed below herein. The granulated products prepared by methods disclosed herein are preferably characterized as being free-flowing, and can be of various particle sizes, e.g. from about 0.1 mm to 2 mm or larger.

As used herein, a “lecithin-based” or “lecithin-containing” “material”, “mixture” or “solid” refers to a substantially solid material containing some amount of lecithin, which material is to be granulated. The amount of lecithin is preferably at least 30% by weight of the material, and it may be up to 100% lecithin (i.e. pure lecithin). In various embodiments, for example, the amount of lecithin in the material to be granulated is at least 40%, at least 50%, at least 60%, at least 70%, at least 80%, at least 90%, or at least 95% by weight. The lecithin in the material to be granulated is typically deoiled lecithin powder.

A “processing agent”, as used herein, refers to a material employed to facilitate the granulation process, such as a binder or antisticking agent.

Other non-lecithin materials which may be included in the lecithin-containing material to be granulated (or in a binder solution during fluid bed granulation) may be referred to herein as additional or further “ingredients”, “components”, or “substances”. These ingredients can vary widely and may include any material that is desired to be combined with lecithin in a granular product. Examples include foodstuffs, pharmaceuticals, nutraceuticals, flavors, colorings, pigments, etc.

A “nutraceutical” includes any food, food supplement or dietary supplement that is believed to provide health or medical benefits. A “pharmaceutical” includes any therapeutically active or preventative substance.

III. Granulation Processes

According to one aspect of the invention, granulation processes are provided for preparing lecithin or lecithin-containing products in granular form. The processes are especially useful for granulation of pure (100%) lecithin powder, and for granulation of lecithin-containing materials having a high proportion of lecithin, e.g. ≧30%≧50%, ≧65%, or ≧80% by weight, including 99% or 99.9% by weight or higher of lecithin.

The granulation processes are suitable for incorporating a variety of additional substances, in either solid or solution form, into the lecithin-containing granules, if desired. Granules can be provided in a variety of particle sizes.

The processes disclosed herein are highly efficient relative to the existing acetone wet granulation process conventionally used for “granulation” of lecithin. The acetone wet granulation process generally produces only a fraction (e.g. about 15%-50% of starting material) of actual granular material, accompanied by large quantities of fine, powdery material.

In the currently provided processes, as described further below, a binder is added to lecithin powder or the desired lecithin-containing material. In roller compaction granulation, an anti-sticking agent is also used. Each of these processing agents is added in an amount at least sufficient to prevent excessive compaction or sticking during or after processing, as described further below.

Various types of granulators, such as, for example, knife granulators, fluid bed granulators, and roller compaction granulators, can be used. Roller compaction and fluid bed granulation are discussed in more detail below.

A. Roller Compaction Granulation

The roller compaction granulation process provided herein combines lecithin and/or other ingredients, in substantially dry form, with a dry binder and antisticking agent, to form granular lecithin or lecithin-containing products. The starting material is typically lecithin powder, with or without additional ingredients. The product obtained consists of non-sticky, preferably free-flowing granules of lecithin and/or incorporated substances.

Any of various binder materials known in the art can be used in the process. For preparation of consumable products, a food grade binder is used. These include, for example, sugars, such as mannitol, sorbitol, or xylitol, starches, such as maltodextrin, lactates, soy proteins, and hydrophilic polymers, such as cellulose ethers, gums such as guar gum, agar, and polylactates. In preferred embodiments, the binder is a cellulosic polymer, preferably a cellulose ether, such as methyl cellulose, ethyl cellulose or hydroxypropyl methylcellulose. A variety of cellulose ethers, of different composition and molecular weights, are available, for example, from Dow Chemical Co. under tradenames such as Methocel®.

The binder is added in an amount sufficient to prevent excessive compaction, sticking or brittleness during or after processing. Preferably, the binder is present at a level of 0.1-5% by weight, more preferably 0.2-1.5% by weight, relative to the lecithin-containing material to be granulated. In further embodiments, the binder is present at 0.5-1.0%, preferably 0.5-0.75%, and more preferably about 0.5% by weight (e.g. 0.5 g binder/kg of lecithin-containing material), relative to the lecithin-containing material. The use of the above-referenced binders at these levels is believed to reduce the deleterious effects of moisture on the processing of the hygroscopic lecithin powder, and to result in good throughput and reduced brittleness of the compacted sheet.

Any of various antisticking agents known in the art can be used, to prevent sticking of the material to surfaces of the granulator. For preparation of consumable compositions, a food grade antisticking agent, such as food grade (vegetable origin) magnesium stearate, is used. Other examples include other metal stearates, such as calcium or zinc stearate, tricalcium phosphate, silicon dioxide, calcium silicate, tricalcium phosphate, and simethicone. The antisticking agent is preferably present at a level of 0.1-5% by weight, more preferably 0.5-1.5% by weight, relative to the lecithin-containing material.

The lecithin-containing material to be granulated comprises lecithin, typically in powder form, and, optionally, other ingredients, such as those described in Sections IV and V below. In various embodiments, the lecithin-containing material to be granulated contains about 30-100% by weight lecithin, about 50-99.9% by weight lecithin, or about 75-95% lecithin. In preferred embodiments, lecithin powder having a fine particle size, e.g. about 0.5 mm or smaller, is employed. An example is Lecigran™ 5750, employed in the Examples below, which has a particle size of about 0.25-0.3 mm or less.

The ingredients are combined, in dry form, with the binder and antisticking agent, preferably using a V-blender or other conventional method for mixing dry ingredients, prior to introducing the mixture into the feed inlet of a roller compaction granulator.

In one embodiment of the method, the milling unit and/or the roller of the granulator are cooled, which has been found by the present authors to prevent or reduce buildup of lecithin or lecithin-containing material within the granulator. Preferably, the milling unit is cooled. The temperature to which the milling unit (and/or roller) is cooled is preferably between 0° C. and about −25° C., and more preferably between about −10° C. and −20° C. Such cooling may be effected in various conventional ways, such as by passing liquid N₂ though the milling unit.

Exemplary process conditions include those provided in Table 2, where granulation of the indicated materials was carried out on a lab scale Vector Fruend model TF 156 roller compaction granulator. Any roller compaction granulator can be used in the process. Process parameters such as feed screw speed, roller speed, roller clearance (shim), and compression pressure generally vary with the equipment used and/or the particular mixture being processed. These parameters are adjusted as needed, in accordance with procedures known by those skilled in the art, to maintain smooth feeding of the material and to minimize buildup within the granulator.

The compacted material is ground or chopped within the granulator to particles of the desired size range. As noted above, a variety of sizes can be produced, e.g. by employing different mesh size screens. For example, particle sizes in the range of 0.8 mm to 1.25 mm (14-25 mesh) are generally suitable for nutraceutical products and other dietary supplements; other size ranges may be preferred for other uses of the granular lecithin products.

Typically, the granular particles are sorted by screening, and particles which are not in the desired size range can be recycled through the process, with larger granules recycled to the granulation chamber (for further granulation, e.g. grinding or chopping) and smaller granules to the feed hopper (for recompaction). As described in Example 2 below, second-pass granules processed in this manner were found to be visually comparable to those obtained from the first pass.

B. Fluidized Bed Granulation

In another aspect of the invention, a fluidized bed granulator is used for granulation of lecithin or a lecithin-containing material. The fluidized bed granulator employs a binder solution to agglomerate powder particles of a lecithin-containing solid material into larger granules. The lecithin-containing material may be 100% lecithin, typically in powder form, or it may contain additional ingredients, such as those described in Sections IV and V below.

The binder solution may also contain one or more additional components. Substances to be incorporated in significant quantities (e.g. more than about 5% by weight total) are more generally included in the lecithin-containing solid mixture, rather than in the solution.

The lecithin-containing solid material preferably comprises at least about 30% by weight of lecithin. The mixture may contain higher amounts of lecithin, e.g. 50%, 75%, 90%, 95%, or 99% by weight. The “lecithin-containing solid mixture” may also be 100% lecithin, as noted above.

In a top-spray fluidized bed granulator, mixed dry ingredients within the granulator are pre-heated by an upward flow of heated air. Granulation occurs by spraying liquid, atomized by high-pressure air, into the heated fluidized powder from a nozzle positioned above. Generally, at least a portion of the sprayed solvent is immediately lost to evaporation. Such granulators per se are well known in the art.

Any fluidized bed granulator can be used in the process. In the procedures described in Example 1 below, a laboratory scale batch granulator was used. Both batch and continuous granulators can be used.

In accordance with the invention, a lecithin-containing solid mixture is granulated in a fluidized bed granulator, using a binder solution having a viscosity which is effective to provide efficient spraying and atomization of the solution. The viscosity may be 50 to 1000 cps (centipoise), is generally in the range of 100 to 500 cps, and is preferably in the range of 100 to 300 cps. The binder solution may be an aqueous solution, or it may employ another solvent of sufficient volatility in which the binder is soluble. Typical examples of such solvents include acetone and C1-C4 alcohols, preferably lower alcohols such as methanol, ethanol, or isopropanol. Mixtures of such solvents with water may also be used.

Any of various binder materials known in the art can be used in the process. For preparation of consumable products, a food grade binder is used. These include, for example, sugars, e.g. mannitol, sorbitol, or xylitol, starches, such as maltodextrin, lactates, soy proteins, and hydrophilic polymers, such as cellulose ethers, gums such as guar gum, agar, and polylactates. In preferred embodiments, the binder is a cellulose ether, such as methyl cellulose or hydroxypropyl methylcellulose. In one embodiment, the binder is methylcellulose. A variety of cellulose ethers, of different composition and molecular weights, are available, for example, from Dow Chemical Co. under the tradename Methocel®.

The binder is added in an amount sufficient to prevent excessive compaction or sticking during or after processing. Particle size of the granules can be adjusted by adjusting the quantity and/or droplet size of the binder solution.

Preferably, the binder solution includes an amount of binder (solids) which is up to 5% by weight, e.g. 0.1-5% by weight, more preferably 0.2-1.5% by weight, relative to the weight of the lecithin-containing material. In selected embodiments, the binder is present at 0.5-1.5% percent by weight (solids) of the lecithin-containing material. For example, in selected procedures described in the working examples below, for 1 kg of lecithin-containing powder to be granulated, 200 g of 5% binder solution (i.e. 10 g solids, or 1% by weight relative to the lecithin-containing material) was employed.

The solvent content of the binder solution is preferably minimized, to reduce liquid content during the granulation process. However, the viscosity of the solution must also be low enough to provide efficient spraying and atomization of the solution, as discussed above. Accordingly, lower viscosity binders are generally preferred. For example, aqueous solutions of Methocel® A4M, or a polymer of similar size and composition, in a concentration range of 2-8%, preferably 3-6%, and more preferably 4-5% by weight, are generally of suitable viscosity.

When the binder solution also includes an additional component to be incorporated into the lecithin granules, the solution preferably includes an amount of such component which is 0.001-1% by weight, relative to the total weight of the lecithin or lecithin-containing solid material. In selected embodiments, the component is present at 0.01-1.0% percent by weight (solids) of the lecithin or lecithin-containing solid material. In all cases, the viscosity of the binder solution is preferably in the ranges cited above, more preferably about 300 cp or less, and most preferably about 100 to 300 cps.

Air flow rate should be sufficient to fluidize the solid particles such that binder is uniformly applied to the particles. Air flow rates of about 25-50 ft³/min, more preferably 30-45 ft³/min, are generally suitable.

Air flow rate and temperature are maintained such that moisture introduced by the binder solution, as well as moisture that may be present within the lecithin material itself, is rapidly, preferably instantaneously, vaporized upon contact of the fluidized solid powder with the solution components. The inlet temperature is preferably at least 40° C., more preferably at least 50° C. or at least 60° C., typically up to about 80° C. In some cases, temperatures up to about 100° C. may be used. Preferred ranges include 45-70° C., more preferably 55-65° C.

The granules are subsequently dried with heated air. The outlet temperature of the granulator is generally maintained at about 35-60° C., although higher temperatures, up to about 70° C., may be used. Preferably, the difference between inlet and outlet temperature is about 30° C. or less, more preferably about 15° C. or less.

Top-spray fluid bed granulation was used to granulate several nutraceutical mixtures containing lecithin as the major ingredient, as shown in Example 1 below. The process provided fine granules with excellent non-sticking and flow characteristics and appearance.

A fluidized bed granulator may also be used for coating a further ingredient onto lecithin-containing granules formed, as described above, by fluidized bed granulation or by roller compaction granulation. This process is useful when it is desired to include a component as a coating rather than dispersed throughout the granule. Accordingly, the granules are further processed in a fluidized bed granulator, at a temperature of at least 40° C., with a solution of the further ingredient. Again, the viscosity of the solution is effective to provide efficient spraying and atomization of the solution, and is preferably 300 centipoise or less. The solution may be an aqueous solution, or it may employ another solvent of sufficient volatility in which the further component is soluble. Typical examples include acetone and alcohols, preferably lower alcohols such as methanol, ethanol, or isopropanol; mixtures of such solvents with water may also be used.

IV. Lecithin-Containing Granular Compositions

In one aspect, the invention provides granular lecithin, as well as granular compositions containing at least 30% by weight, e.g. 50%, 75%, 90%, 95%, or 99% by weight, of lecithin. In various other embodiments, the granular compositions may include at least 40%, at least 50%, at least 60%, at least 70%, at least 80%, at least 90%, or at least 95% lecithin by weight. Such granular compositions can be prepared from lecithin (e.g. deoiled lecithin powder), with or without incorporation of additional components, by the granulation methods described above.

The lecithin granules, or lecithin-containing granules, are preferably free-flowing. The flowable characteristic of the granules can be evaluated, in one manner, by measurement of the angle of repose, which is defined as the maximum angle at which a pile of unconsolidated material remains stable. Accordingly, a lower number indicates a more free-flowing composition. Preferably, for the granules disclosed herein, this angle is 65° or less. More preferably, the angle is less than 60°, less than 55°, less than 50°, less than 45°, or less than 40°.

The lecithin or lecithin-containing granules can be provided in various particle sizes. The fluid bed granulation process disclosed herein typically produces fine granules, e.g. less than 1 mm, or less than 0.5 mm, in size. The roller compaction granulation process disclosed herein can produce granules in a large variety of size ranges, which may be determined, for example, by the mesh size used for screening. Such mesh sizes and the corresponding particle size ranges are well known in the art. For example, a mesh size of 14-25 generally corresponds to particles in the size range of about 0.8 to 1.25 mm; a mesh size of 8-12 generally corresponds to particles in the size range of about 1.7 to 2.4 mm; etc.

In accordance with the invention, the granular lecithin or lecithin-containing compositions include a binder, as described above, typically in an amount of about 0.1% to 5% by weight, more preferably 0.2-1.5% by weight, relative to the weight of the lecithin-containing material; that is, relative to the weight of lecithin and/or other incorporated ingredients. The granular compositions may also include an antisticking agent, as described above, which is typically present in an amount of about 0.1% to 5% by weight, more preferably 0.2-1.5% by weight, relative to the weight of lecithin and/or other incorporated ingredients.

In one embodiment, the granular compositions provided herein consist of lecithin and a binder as described above. Such compositions can be prepared by the fluid bed granulation process described above. In another embodiment, the granular compositions provided herein consist of lecithin, a binder as described above, and an antisticking agent as described above. Such compositions can be prepared by the roller compaction granulation process described above. In these compositions, the binder is preferably present in an amount of about 0.1% to 5%, preferably 0.2 to 1.5%, and more preferably 0.5 to 1% by weight, relative to the weight of lecithin. The antisticking agent, when present, is preferably present in an amount of about 0.1% to 5%, preferably 0.2 to 1.5%, and more preferably 0.5 to 1% by weight, relative to the weight of lecithin.

In further embodiments, where additional ingredients are incorporated into the lecithin-containing material to be granulated, and thereby into the granular compositions, the granular compositions provided herein consist of lecithin, the additional ingredient(s), and a binder as described above. Such compositions can be prepared by the fluid bed granulation process described above. In another embodiment, the granular compositions provided herein consist of lecithin, the additional ingredient(s), a binder as described above, and an antisticking agent as described above. Such compositions can be prepared by the roller compaction granulation process described above. In these compositions, the binder is preferably present in the amounts specified above, relative to the weight of lecithin and the additional ingredient(s). The antisticking agent, when present, is preferably present in the amounts specified above, relative to the weight of lecithin and the additional ingredient(s).

When additional ingredients are present, the granular lecithin-containing compositions, exclusive of processing agents (i.e. binder and/or antisticking agent), typically comprise at least 30% by weight of lecithin. In further embodiments, these compositions comprise, exclusive of processing agents, at least 50% by weight, at least 85% by weight, or at least 95% by weight, of lecithin.

As noted above, other non-lecithin materials which may be included in the lecithin-containing material to be granulated (or in a binder solution during fluid bed granulation) can vary widely and may include any material that is desired to be combined with lecithin in a granular product, for any use. Examples include foodstuffs, pharmaceuticals, nutraceuticals, flavors, colorings, pigments, etc. The incorporation of nutraceuticals into the lecithin-containing granules is discussed in more detail below.

V. Granular Lecithin-Based Nutraceutical Compositions

Lecithin is widely used in combination with other consumable substances in the food and pharmaceutical industries, by virtue of its properties as a solubilizer, dispersant, taste masking ingredient, etc. Lecithin also possesses beneficial therapeutic properties in its own right. For example, lecithin has significant cholesterol lowering and liver protecting properties. Lecithin is also a significant constituent of nervous and brain tissue, and functions as a precursor of the neurotransmitter acetylcholine. It has been shown to increase levels of acetylcholine in specific brain structures, and under certain conditions may enhance cholinergic neurotransmission. Lecithin has therefore been recommended for patients afflicted with neuropsychiatric diseases associated with deficient cholinergic activity, including Huntington's chorea, schizophrenia, affective illness, and senile dementia of the Alzheimer type.

In accordance with one aspect of the invention, lecithin is combined with other consumable substances, such as those described below, to form a granular composition. Such compositions may be prepared by the granulation methods described above, by incorporating the consumable substance, such as a nutraceutical, into the lecithin-containing material to be granulated. The lecithin-containing granules produced as disclosed herein are typically free-flowing and non-sticky and have good organoleptic properties; e.g. good appearance and mouth feel.

In preferred embodiments, the consumable substance is an active ingredient, such as a nutraceutical substance. Other consumable active ingredients can also be used, alone or in combination with nutraceuticals or other additives. These include, for example, poorly bioavailable substances in general, including foods, feeds, and pharmaceuticals, to improve the bioavailability of these substances; poorly soluble or poorly dispersible substances in general, again including foods, feeds, and pharmaceuticals, to improve the solubility and/or dispersibility of these substances; bitter or otherwise bad tasting substances in general, to mask bitterness or bad taste; substances which could affect the stomach wall (e.g., drugs like aspirin, indomethacin, and other NSAIDs), to reduce irritation; and proteins, including hydrolyzed proteins and enzymes, to improve physiological performance of these substances or to protect them from environmental influences, including destructive effects of the digestive system. Inactive consumable ingredients such as flavors, sweeteners, and colors can also be included.

In one embodiment, at least one active ingredient in the composition is a nutraceutical substance. Nutraceuticals which may be incorporated into lecithin-containing granules, in accordance with the invention, include, for example, vitamins, amino acids, enzymes, minerals, trace elements, glucosamine, chondroitin, pectin, flavonoids, isoflavonoids, lignans, camosine, polyunsaturated fatty acids, antioxidants, such as anthocyans, proanthocyanidines, and carotinoides (e.g. lutein and lycopene, as noted above, as well as astaxanthin or zeaxanthin), phytin, phytic acid, policosanols, policosanoic acids, montanic acids, pyruvates, and various other phospholipids, such as phosphatidylserine or phosphatidylethanolamine. Also included are various plant extracts, such as green tea extract or artichoke leaf extract. Other plant extracts include those disclosed, for example, in U.S. Patent Appn. No. 2003/0104076, which is incorporated herein by reference. Vitamins include especially Vitamin A, B-complex, C, D, E, K, β-carotene, nicotinamide, folic acid, and NADH, as well as coenzyme Q10 and L-carnitine, noted above. Minerals and trace elements include especially calcium, magnesium, sodium, potassium, chromium, iodine, manganese, copper, iron, zinc, vanadium, phosphorus, and selenium, in physiologically acceptable form.

In the exemplary nutraceutical compositions described below, nutrients were selected to support the physiological activity of lecithin.

(a) Lecithin/Phytosterol(s) and/or Phytostanols

Phytosterols and phytostanols have been shown to reduce total cholesterol and LDL levels in serum, as well as having antitumor effects (see e.g. W. H. Ling et al., “Dietary phytosterols: a review of metabolism, benefits and side effects”, Life Sci. 57(3): 195-206, 1995). Combining these cholesterol-lowering nutrients allows a reduced daily dosage of phytosterols and/or phytostanols (see e.g. R. E. Ostlund et al., “Sitostanol administered in lecithin micelles potently reduces cholesterol absorption in humans”, Am. J. Clin. Nutr. 70:826-831, 1999). In a preferred embodiment, the phytosterol component is a phytosterol mixture provided by Cargill, Inc. under the tradename Corowise™. The two components (that is, lecithin and the phytosterol/phytostanol component) are preferably present in a ratio of 5:1 (lecithin:phytosterol/phytostanol) or greater; i.e. 1 g of phytosterol/phytostanol in 6 g of granules. Other ratios may include, for example, 7:1, 10:1, 20:1, 50:1, or 100:1.

(b) Lecithin/Coenzyme Q10

Coenzyme Q10 is known to be highly concentrated in heart muscle cells, which have a high energy requirement. Congestive heart failure has been correlated with low blood and tissue levels of CoQ10. Cholesterol reduction and energy support for the heart muscle is a useful combination for prevention of cardiovascular problems. The Q10 component is preferably present at a level of about 0.4% or less; for example, 1, 5, 10, 15, 20, or 25 mg Q10 in 6 g of lecithin-containing granules.

(c) Lecithin/Lycopene

This combination provides a powerful antioxidant with a balanced combination of phospholipids. The lycopene component is preferably present at a level of about 0.1% or less; for example, 0.5, 1, 2, 2.5, 3, 4, or 5 mg lycopene in 6 g of lecithin-containing granules.

(d) Lecithin/Lutein

This combination provides improved bioavailability of lutein for prevention of AMD (age-related macular degeneration). The lutein component is preferably present at a level of about 0.1% or less; for example, 0.5, 1, 2, 2.5, 3, 4, or 5 mg Q10 in 6 g of lecithin-containing granules.

(e) Lecithin/Creatine

In this preparation, lecithin, which functions as an acetylcholine (neurotransmitter) precursor, is combined with creatine to improve muscular contractability and performance. The two components (that is, lecithin and creatine, which may be provided as the monohydrate) are preferably present in a ratio of 2:1 (lecithin/creatine) or greater. Other ratiosmay include, for example, 3:1, 4:1, 5:1, 7:1, 10:1, 20:1, 50:1, or 100:1.

(f) Lecithin/L-Carnitine

This preparation is useful for promotion of fat breakdown and energy production. The two components (that is, lecithin and L-carnitine) are preferably present in a ratio of 2:1 or greater. Other ratios may include, for example, 3:1, 4:1, 5:1, 7:1, 10:1, 20:1, 50:1, or 100:1.

In each of formulations (a)-(f) above, a therapeutic daily dose would consist of about 6 g of lecithin-containing granules containing the minimum ratios of lecithin/nutraceutical component noted above. Administration as a dietary supplement would employ a lower daily amount of the non-lecithin component.

### EXAMPLES

The following examples are intended to illustrate but not to limit the invention.

Granules of lecithin and of various lecithin-containing materials, typically nutraceutical mixtures, were prepared using roller compaction granulation or top spray fluidized bed granulation.

Materials and Methods

The lecithin used as starting material in these procedures was Lecigran™ 5750 produced by Riceland Foods, Inc. This is a deoiled lecithin powder having a particle size of about 0.25-0.31 mm or less.

The phytosterols used were a powdered mixture of vegetable oil phytosterols provided by Cargill, Inc. under the tradename Corowise™, having as major components sitosterol (ca. 40-58%), campesterol (ca. 20-28%) and stigmasterol (ca. 14-23%). The creatine monohydrate used was Creapure™ supplied by Degussa AG. The L-carnitine was a crystalline product supplied by Lonza Inc. The coenzyme Q10 used was a powdered product supplied by Asahi Kasei Pharma. The lutein used was FloraGlo® Lutein (20%) liquid in corn oil, supplied by Kemin Industries. The lycopene used was Tomat-O-Red® 10% CWD from Lycored Natural Products Industries Ltd.

The binders employed were food grade methyl cellulose polymers provided by Dow Chemical Co., designated Methocel® A4C and Methocel® A4M. The binder can be used as an aqueous solution (top spray fluidized bed granulator, Example 1) or as a dry powder (roller compaction granulator, Example 2). A food grade (vegetable origin) magnesium stearate was used as a non-sticking agent in the roller compaction granulation process. A laboratory scale batch granulator (Vector model FL M-1) was used for the fluidized bed granulation runs. The roller compaction granulator used was a Vector Fruend model TF 156 laboratory scale granulator.

### Example 1

### Fluidized Bed Granulation

Granulation of solid material (designated “Lecithin Containing Material” in the Table below) was carried out in a top-spray fluidized bed granulator (a Vector F1-M-1 Fluid Bed Processor). A summary of several granulation runs is provided in Table 1. Binder designations refer to Methocel® binders; see Materials and Methods, above.

TABLE 1

| Lecithin | | | | | | | |
|---|---|---|---|---|---|---|---|
| Containing | | Solution | Solution | Inlet | Outlet | | |
| Material | Binder | Feed Rate | Loading | Temp. | Temp. | Air Flow | Angle of |
| (1 kg) | Solution | (g/min) | (g) | (° C.) | (° C.) | (ft³/min) | Repose |
| 100% lecithin | 1% A4M | 10 | 110 | 51 | 37 | 32 | n.d. |
| 100% lecithin | 1% A4M | 5 | 160 | 55 | 55 | 30 | 65° |
| 2:1 lecithin:creatine.H₂O | 1% A4M | 5 | 250 | 71 | 48 | 36 | 51° |
| 2:1 lecithin:creatine.H₂O | 5% A4M | 5 | 200 | 88 | 61 | 45 | 42° |
| 2:1 lecithin:L-carnitine | 5% A4M | 5 | 200 | 100 | 70 | 36 | 57° |
| 5:1 lecithin:phytosterols | 5% A4M | 5 | 200 | 100 | 71 | 40 | 54° |

Fine granular products were obtained, having good flow characteristics and mouth feel.

The angle of repose was determined for the products of runs 2-6. Angle of repose is defined as the maximum angle at which a pile of unconsolidated material remains stable. Accordingly, a lower number indicates a more free-flowing composition. The angle of repose for lecithin powder is generally about 720.

### Example 2

### Roller Compaction Granulation

In a first series of experiments, roller compaction granulation was carried out using Methocel® A4C as binder, at a level of 1% by weight of the lecithin or lecithin-containing material, and magnesium stearate as antisticking agent, at a level of 0.5-1% by weight of the lecithin or lecithin-containing material. The lecithin used was Lecigran™ 5750, as described above under Materials and Methods. In all but one of these procedures, an additional (nutraceutical) component was included in the material to be granulated.

The materials to be granulated, binder, and antisticking agent were combined and mixed in a V blender (Patterson Kelley). A Vector Fruend model TF156 roller compaction granulator was used for granulation. A summary of several granulation runs is provided in Table 2.

TABLE 2

| | Dry | Antisticking | Feed | | | | |
|---|---|---|---|---|---|---|---|
| | binder | agent | Screw | Roller | Comp. | Roller | Milling |
| | (A4C) | (Mg stearate) | Speed | Speed | Pressure | clearance | screen |
| Ingredients | (wt %) | (wt %) | (rpm) | (rpm) | (psi) | (in) | (mesh) |
| 100% lecithin | 1 | 0.5 | 12 | 4 | 600 | .01 | 4 |
| 2:1 lecithin:creatine.H₂O | 1 | 1 | 27.3 | 3.8 | 1000 | .01 | 8 |
| 2:1 lecithin:creatine.H₂O | 1 | 1 | 20.6 | 3.8 | 2500 | .05 | 8 |
| 2:1 lecithin:creatine.H₂O | 1 | 1 | 20.8 | 3.8 | 500 | .01 | 8 |
| 2:1 lecithin:L-carnitine | 1 | 1 | 19.7 | 3.8 | 2500 | .01 | 8 |
| 2:1 lecithin:L-carnitine | 1 | 1 | 19.7 | 3.8 | 2500 | .0.05 | 8 |
| 2:1 lecithin:L-carnitine | 1 | 1 | 24.4 | 3.8 | 600 | .01 | 8 |
| 5:1 lecithin:phytosterols | 1 | 1 | 19.9 | 3.8 | 2500 | .0.05 | 8 |

The granules obtained had excellent non-sticking and flow characteristics, with good mouth feel and taste.

Additional runs are summarized in Table 3. In these runs, the use of 0.5 wt % or 0.75 wt % of binder and antisticking agent was found to give results comparable to those obtained using 1.0 wt % of these additives.

In the granulation runs summarized in Table 3, the milling unit was cooled, using liquid nitrogen, to a temperature of about −12° C. In run 6, the compacting roll was also cooled, to about −16° C. Cooling of the milling unit, in particular, was found to reduce buildup of lecithin in the milling unit and facilitate continuous operation.

“L5750” refers to Lecigran™ 5750, produced by Riceland Foods, Inc., as described above, which performed well in all of these procedures. In several of the runs, as indicated, 0.34 weight % of a vitamin supplement was blended into the lecithin powder using a V-blender, blending for about 10 minutes, prior to granulation.

In each case, feed screw speed was as indicated, and roller speed was 5 rpm. Feed rates of about 20-30 kg/hr, which is near the maximum indicated for this equipment, were generally sustained.

Granules in the target size range (0.8 to 1.25 mm), as determined by the screen mesh size, were obtained in about 40-45% yield in the first pass from runs 6-8. As noted above, non-target size material can be recycled. Two runs, as indicated, employed 55% recycled (2^{nd} pass) material (larger than 14 mesh (1.4 mm) or smaller than 25 mesh (0.7 mm)). The product of these runs was visually comparable to first-pass granulated product. In all cases, the granulated product had good non-sticking and flow characteristics.

TABLE 3

| | | | | Antisticking | Feed | | |
|---|---|---|---|---|---|---|---|
| | Starting | | Binder | agent | Screw | Roller | Milling |
| | Material | Vitamins | (A4C) | (Mg stearate) | Speed | clearance | screen |
| Run | Lecithin | (wt %) | (wt %) | (wt %) | (rpm) | (in) | (mesh) |
| 1 | L5750 | — | 1.0 | 1.0 | 58.8 | 0.06 | 8 |
| 2 | L5750 | — | 1.0 | 1.0 | 58.8 | 0.06 | 8 |
| 3 | L5750 | — | 0.75 | 0.5 | 59 | 0.05 | 8 |
| 4 | L5750 | — | 0.5 | 0.5 | 59 | 0.05 | 8 |
| 5 | L5750 | — | 0.33 | 0.5 | 60 | 0.05 | 6 |
| 6 | L5750 | — | 0.75 | 0.5 | 59.7 | 0.05 | 10 |
| 7 | L5750 | 0.34 | 1.0 | 1.0 | 11.5 | 0.05 | 10 |
| 8 | L5750 | 0.34 | 0.66 | 0.5 | 11.5 | 0.05 | 10 |
| 9 | L5750 | — | 0.66 | 0.5 | 11.5 | 0.05 | 10 |
| 10 | L5750 45% | 0.34 | 0.66 | 0.5 | 11.5 | 0.05 | 10 |
| | 2^{nd} pass 55% | | | | | | |
| 11 | L5750 45% | 0.34 | 0.66 | 0.5 | 12 | 0.05 | 10 |
| | 2^{nd} pass 55% | | | | | | |

While the invention has been described with reference to specific methods and embodiments, it will be appreciated that various modifications may be made without departing from the invention.

1. A method of producing a granular lecithin-containing composition, the method comprising:
processing in a roller compaction granulator: (i) a solid comprising at least 30% by weight lecithin and an optional further component, with (ii) a binder material, in an amount of about 0.1% to 5% by weight relative to said solid, and (iii) an antisticking agent, in an amount of about 0.1% to 5% by weight relative to said solid.

2. The method of claim 1, wherein each of said binder material (ii) and antisticking agent (iii) is present in an amount of about 0.2% to 1.5% by weight, independently, relative to said solid (i).

3. The method of claim 1, wherein said binder material is selected from the group consisting of sugars, starches, lactates, soy proteins, and hydrophilic polymers.

4. The method of claim 3, wherein said hydrophilic polymer is selected from cellulose ethers, gums, and polylactates.

5. The method of claim 4, wherein said binder material is a cellulose ether.

6. The method of claim 4, wherein said cellulose ether is present in an amount of about 0.5% to 1% by weight relative to said solid (i).

7. The method of claim 1, wherein said antisticking agent is selected from the group consisting of silicon dioxide, calcium silicate, tricalcium phosphate, calcium stearate, zinc stearate, and magnesium stearate.

8. The method of claim 7, wherein said antisticking agent is magnesium stearate.

9. The method of claim 7, wherein said magnesium stearate is present in an amount of about 0.5% to 1% by weight relative to said solid (i).

10. The method of claim 1, wherein said solid (i) contains at least 50% by weight lecithin.

11. The method of claim 10, wherein said solid contains at least 75% by weight lecithin.

12. The method of claim 11, wherein said solid is 100% lecithin.

13. The method of claim 1, wherein said lecithin is in powder form.

14. A granular lecithin-containing composition produced by the method of claim 1.

15. The composition of claim 14, comprising 30-99% by weight lecithin, a binder, and an antisticking agent.

16. The composition of claim 15, comprising 50-99% by weight lecithin.

17. The composition of claim 16, comprising 75-99% by weight lecithin.

18. A method of producing a granular lecithin-containing composition, the method comprising:
processing in a fluidized bed granulator, at an inlet temperature of at least 40° C., (i) a solid comprising at least 30% by weight lecithin and an optional further component, with (ii) a solution of a binder material, said solution having a viscosity effective to provide efficient spraying and atomization of the solution.

19. The method of claim 18, wherein said viscosity is 100 to 300 centipoise.

20. The method of claim 18, wherein the solution comprises a solvent selected from the group consisting of water, acetone, and C1-C4 alcohols.

21. The method of claim 20, wherein said solvent is water.

22. The method of claim 18, wherein at least one of said solid and said solution contains one or more further components.

23. The method of claim 18, wherein said binder material is selected from the group consisting of sugars, starches, lactates, soy proteins, and hydrophilic polymers.

24. The method of claim 23, wherein said hydrophilic polymer is selected from cellulose ethers, gums, and polylactates.

25. The method of claim 24, wherein said binder material is a cellulose ether.

26. The method of claim 25, wherein said cellulose ether is methyl cellulose or hydroxypropyl methylcellulose.

27. The method of claim 18, wherein the inlet temperature is at least 50° C.

28. The method of claim 18, wherein the inlet temperature is about 40-100° C.

29. The method of claim 28, wherein the inlet temperature is at about 50-90° C.

30. The method of claim 18, wherein said solid (i) contains at least 50% by weight lecithin.

31. The method of claim 30, wherein said solid (i) contains at least 75% by weight lecithin.

32. The method of claim 18, wherein said solid (i) is 100% lecithin.

33. A granular lecithin-containing composition produced by the method of claim 18.

34. The composition of claim 33, comprising 30-99.9% by weight lecithin and a binder.

35. The composition of claim 34, comprising 50-99.5% by weight lecithin.

36. The composition of claim 35, comprising 75-99.5% by weight lecithin.

37. A granular lecithin-containing composition, comprising:
(i) at least 90% by weight of a preparation comprising lecithin and an optional further component, wherein said preparation contains at least 30% lecithin by weight;
(ii) a binder material, in an amount of about 0.1% to 5% by weight relative to (i), and, optionally,
(iii) an antisticking agent, in an amount of about 0.1% to 5% by weight relative to (i).

38. The composition of claim 37, wherein the preparation is at least 50% by weight lecithin.

39. The composition of claim 37, wherein the preparation is at least 75% by weight lecithin.

40. The composition of claim 37, wherein the preparation is 100% lecithin.

41. The composition of claim 37, wherein said antisticking agent is not present.

42. The composition of claim 40, wherein said antisticking agent is not present.

43. The composition of claim 42, wherein said binder material (ii) is present in an amount of about 0.5% to 2.5% by weight relative to said lecithin.

44. The composition of claim 43, wherein said binder material (ii) is present in an amount of about 0.5% to 1% by weight relative to said lecithin.

45. The composition of claim 44, wherein said binder is selected from the group consisting of sugars, starches, lactates, soy proteins, and hydrophilic polymers.

46. The composition of claim 45, wherein said hydrophilic polymer is selected from cellulose ethers, gums, and polylactates.

47. The composition of claim 46, wherein said binder is a cellulose ether.

48. The composition of claim 37, wherein said antisticking agent is present.

49. The composition of claim 40, wherein said antisticking agent is present.

50. The composition of claim 49, wherein said antisticking agent is present in an amount of about 0.5% to 1.5% by weight relative to said lecithin.

51. The composition of claim 49, wherein said antisticking agent is selected from the group consisting of silicon dioxide, tricalcium phosphate, calcium silicate, calcium stearate, zinc stearate, and magnesium stearate.

52. The composition of claim 51, wherein said antisticking agent is magnesium stearate.

53. The composition of claim 37, which consists of (i) lecithin and (ii) said binder material, which is present in an amount of about 0.1% to 5% relative to said lecithin.

54. The composition of claim 37, which consists of (i) lecithin; (ii) said binder material, which is present in an amount of about 0.1% to 5% by weight relative to said lecithin; and (iii) said antisticking agent, which is present in an amount of about 0.1% to 5% by weight relative to said lecithin.

55. The composition of claim 37, wherein said further component is present and is a nutraceutical component.

56. The composition of claim 55, wherein the nutraceutical component is selected from the group consisting of vitamins, phytosterols, phytostanols, creatine, L-carnitine, coenzyme Q10, lycopene, and lutein.
